(19) United States
(12) Patent Application Publication
(10) Pub. No.: US 2023/0309618 A1
(43) Pub. Date: Oct. 5, 2023

(54) DEVICES AND METHODS FOR VAPORIZATION OF LIQUIDS AND SOLIDS

(71) Applicant: Vuber Technologies LLC, Seattle, WA (US)

(72) Inventor: David Crowe, Lake Forest Park, WA (US)

(21) Appl. No.: 18/205,589

(22) Filed: Jun. 5, 2023

Related U.S. Application Data
(63) Continuation-in-part of application No. 16/878,639, filed on May 20, 2020, now Pat. No. 11,707,090.
Application No. 16/878,639 is a continuation-in-part of application No. 15/860,641, filed on Jan. 2, 2018, now Pat. No. 10,701,977.
(60) Provisional application No. 62/543,316, filed on Aug. 9, 2017.
(60) Provisional application No. 62/593,141, filed on Nov. 30, 2017.

(51) Int. Cl. A24F 40/44; A24F 40/10; A24F 40/42; A24F 40/46; A24F 40/48; A24F 40/57; A24F 40/60; A24F 40/90; H05B 3/22

(52) U.S. Cl. CPC A24F 40/44; A24F 40/10; A24F 40/42; A24F 40/46; A24F 40/48; A24F 40/57; A24F 40/60; A24F 40/90; H05B 3/22; A61M 11/042

(57) ABSTRACT

The present invention discloses an atomizer. The atomizer includes a concentrate reservoir volume that is in fluid communication with a concentrate vaporization assembly. The concentrate vaporization assembly includes a frit adapted to absorb concentrate from the concentrate reservoir volume. The concentrate vaporization assembly further includes a heating element adapted to heat the frit and absorbed concentrate. The atomizer further includes a vapor collection and discharge assembly including a vapor accumulation chamber in fluid communication with the frit and a vapor evacuation channel in fluid communication with the vapor accumulation chamber and in fluid communication with an egress port. The heating element is activated by a user control of a switch on a battery, which causes the concentrate contained within the frit filter to vaporize, and the user inhales resulting vapor by inhaling at the egress port of the vapor evacuation channel.

### CROSS-REFERENCE TO RELATED APPLICATIONS

The present application is a Continuation-in-Part of the U.S. patent application Ser. No. 16/878,639 filed May 20, 2020 entitled “Permeable Element Based Vaporization Process and Device,” which is a Continuation-in-Part of U.S. patent application Ser. No. 15/860,641 filed Jan. 1, 2018 entitled “Permeable Element Based Vaporization Process and Device,” which claims the benefit of U.S. Provisional Application 62/543,316 filed Aug. 8, 2017, entitled “Vaporization Device and Process,” and U.S. Provisional Application 62/593,141 filed Nov. 29, 2017, also entitled “Vaporization Device and Process,”, which are both incorporated herein by reference in their entirety as if fully set forth herein.

### FIELD OF THE DISCLOSURE

The present invention pertains to the field of devices and methods for vaporization, specifically focusing on the efficient and controlled transformation of both liquids and solids into vapor or gas states. The inventions within this field offer novel approaches and advancements that significantly improve the vaporization process across various industries.

### BACKGROUND

Cannabis and tobacco have long been used recreationally and medicinally, with smoking being the traditional and prevalent means for consumption of both. A variety of other means for consumption currently exist, while new consumption means are continually being developed.

Vaporization has gained prevalence as a means for consumption. Vaporization differs from smoking in that the cannabis or tobacco, extracts thereof, or synthesized nicotine or cannabinoid concentrates are merely heated to the point of vaporization, rather than combusted. Vaporization ideally produces an inhalable vapor without producing smoke. Vaporization is a highly controllable process, wherein the amount of heating applied to either the plant or concentrate can be controlled precisely, and the size of the resulting dose of medicament is much more predictable than the size of a dose taken through smoking. Vaporization differs from smoking in that the raw plant or concentrate is heated to a temperature high enough to volatilize the medicament into vapor but low enough to avoid combustion. Combustion products and byproducts, such as smoke and NOR, may be undesirable for consumption for a variety of reasons, including health effects and flavor preference. Vaporization optimally produces no smoke, and the vapor will exhibit a complete absence of any associated burnt flavor.

Vaporizers adapted for use with concentrate typically rely on an ohmic resistive heating element that is either proximal to or in direct contact with the concentrate to be vaporized. Although the temperature and heat output of the heating element is controllable to some degree and is generally calibrated for a desired vapor production, the design inherently produces uneven heating of concentrate. This uneven heating creates some degree of micro localized concentrate burning with resulting smoke and associated burnt flavor. The presence of a burnt flavor can be exacerbated through improper vaporizer operation. Because smoke and burnt flavors are dominant and difficult to mask, even very small degrees of localized burning can produce a persistent burnt flavor.

A need exists for a vaporizer that is substantially resistant to producing smoke or any accompanying burnt flavor.

### SUMMARY

The object of the present invention is to provide an improved concentrate vaporization method that is resistant to localized concentrate burning and an associated vaporizer device adapted for performing said method.

The present invention is a method for vaporizing concentrate that will substantially eliminate general or localized burning of concentrate during the vaporization process and a device adapted for carrying out said method. The vaporization method is based on heating concentrate that has been absorbed into a frit, preferably a fritted glass. Fritted glass is characterized by open-pore interstices that allow free passage of fluid through the frit. It is commonly used as a filtering element, particularly in high-temperature applications. It was discovered that concentrate placed in contact with fritted glass is absorbed through capillary action. Although room temperature concentrate may not readily seep fully into fritted glass, as concentrate is heated its viscosity is reduced such that it is readily absorbed by the fritted glass. While glass frits are preferred, other non-porous, heat resistance materials, such as stainless steel, may be substituted for glass. Intrinsically porous materials, such as ceramic are undesirable in this application, as it introduces uncontrolled pore sizes and geometrics, which result in microscopic, localized burning.

Frits has unusual thermal properties stemming from a combination of extremely high surface area relative to volume, permeability, porosity, high internal thermal resistivity due to relatively low conductive area between the individual sintered components that together comprise the frit. Frits differs from other porous solid filters in that they are formed by sintering process in which a plurality of discrete particles is fused through application of heat and pressure. Unexpectedly, when a frit has absorbed concentrate, the frit may be directly exposed to heat sources, including flame or radiant heat, and concentrate contained within the frit will be heated sufficiently to vaporize, but insufficiently to cause any substantial localized burning. Additionally, frits are themselves filters, and micron filter frits, when used in this application, provide the particulate filtration that further improves the quality of produced vapor over traditional vaporization methods.

The invented method of vaporization is therefore to cause a frit to absorb concentrate, to heat said frit and contained concentrate sufficiently to produce vapor while producing extremely limited localized burning to the extent that any associated burnt flavor would be essentially undetectable by the human palate, and to evacuate said vapor.

The invented device is a vaporizer specially adapted to carry out the invented method.

### BRIEF DESCRIPTION OF DRAWINGS

FIG. 1 shows a flow diagram of the method, according to an embodiment of the present invention.

FIG. 2 shows an isometric view of a vaporizer, according to an embodiment of the present invention.

FIG. 3 shows front and section views of an atomizer, according to an embodiment of the present invention.

FIG. 4 shows front and section views of the atomizer and a detail view of the frit and proximal components, according to an embodiment of the present invention.

FIG. 5 shows front and section views of an alternative embodiment of the atomizer and a detail view of the frit and proximal components.

FIG. 6 shows perspective, front, and section views of an alternative embodiment of the frit having an embedded heating element.

### DEFINITIONS

Vapor: Gaseous or suspended liquid condensate suitable for inhalation. Vapor refers to a gaseous state of matter that consists of tiny particles or droplets of a substance, typically in the form of a gas or suspended liquid condensate. It is commonly produced through the process of vaporization, where a substance transitions from its solid or liquid state into the gas phase. Vaporization occurs when sufficient energy is added to the substance, causing its molecules or particles to gain enough kinetic energy to overcome intermolecular forces and escape into the surrounding environment. In the context of inhalation, vapor refers to the vaporized form of a substance that is suitable for inhalation into the respiratory system. This can include both gases and liquids that have been transformed into a gaseous state. The vapor may contain volatile compounds or active ingredients that can be inhaled for therapeutic, recreational, or other purposes.

Vaporize: to produce vapor from a liquid or solid. Vaporize is a process of converting a substance from its liquid or solid state into vapor or gas form. It involves the transformation of molecules or particles of a substance into the gaseous phase by adding sufficient energy to overcome intermolecular forces and enable their escape into the surrounding environment. When a substance is vaporized, its individual molecules or particles gain enough kinetic energy to break free from their solid or liquid arrangement and disperse as a gas. This conversion typically occurs through the application of heat, but other methods like pressure changes or exposure to radiation can also facilitate vaporization.

Vaporizer: Device used to vaporize. The vaporizer is a device designed for the purpose of vaporizing substances, typically liquids or solids, and converting them into vapor or gas form. It is a specialized apparatus that facilitates controlled heating or atomization of the substance, allowing the user to inhale or otherwise utilize the resulting vapor.

Sinter: To fuse constituent solid components into a single solid component through application of heat and pressure. Sintering is a process in which solid components, typically in powder or particulate form, are fused together to form a single solid component. This fusion occurs through the application of heat and pressure, leading to the creation of strong bonds between the particles.

Glass: any solid comprised mostly of vitreous silica. It is characterized by its non-crystalline structure, in which the atoms or molecules are arranged in a disordered manner, lacking long-range periodicity. Glass is typically produced through the rapid cooling or solidification of molten materials, such as silica sand, combined with other additives. This rapid cooling process prevents the formation of a crystalline structure, resulting in an amorphous solid with unique optical, thermal, and mechanical properties. Glass finds widespread use in various applications, including windows, containers, optical lenses, and electronic displays, due to its transparency, durability, and versatility as a material.

Quartz glass: glass comprised of chemically pure vitreous silica. It is produced by melting high-purity silica sand or quartz crystals and then cooling the molten material to form a solid glass. Quartz glass exhibits exceptional thermal stability, high transparency to ultraviolet, visible, and infrared light, low thermal expansion, and excellent resistance to chemical corrosion. Due to its purity and unique properties, quartz glass is widely used in applications that require high thermal resistance, such as laboratory equipment, optical components, semiconductor manufacturing, and high-temperature environments. Its chemically pure composition makes quartz glass highly desirable for applications where impurities can cause unwanted effects or interactions with other materials.

Frit: Permeable vaporization element such as sintered glass or filter of any intrinsically non-porous and heat resistant composition (for example, stainless steel) that is substituted where a frit of glass may otherwise have been used.

Concentrate: Formulation of extracted active ingredients from Cannabis or Tobacco, including cannabinoids such as THC or CBD, alkaloids such as nicotine, or other medicinal or psychoactive compounds, or synthetic versions thereof. Generically, this includes cannabinoid extracts such as oil, wax, budder, shatter, and similar products, as well as nicotine products such as e-juice and similar products.

### DETAILED DESCRIPTION

In the Summary above, Detailed Description, claims below, and accompanying drawings, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used—to the extent possible—in combination with and/or in the context of other particular aspects and embodiments of the invention, and in the invention generally.

The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also contain one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range including that number (which may be a range having an upper limit or no upper limit, depending on the variable being defined). For example, “at least 1” means 1 or more than 1. The term “at most” followed by a number is used herein to denote the end of a range, including that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose limits include both numbers. For example, “25 to 100” means a range whose lower limit is 25 and upper limit is 100, and includes both 25 and 100. Further, in the context of the present disclosure, the terms “frit”, “frit filter”, etc are interchangeably used but should be considered to indentify the similar items. Likewise, the terms such as “heat source”, “heating element”, etc are interchangeably used to and should be considered to identify similar items.

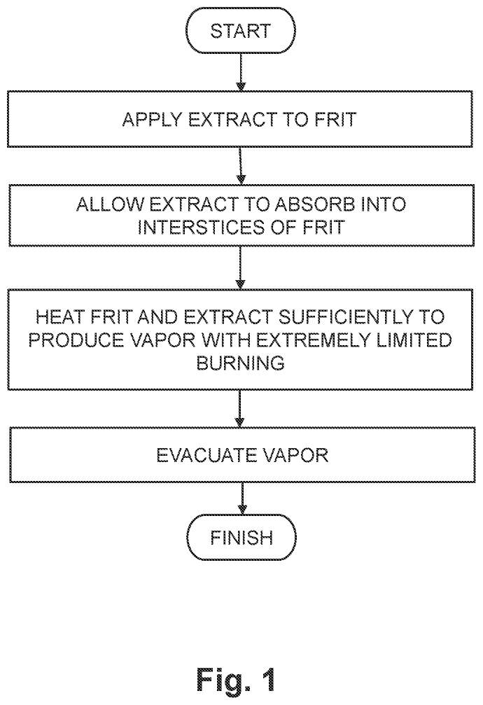

FIG. 1 shows a flow diagram of the invented method, comprised of four steps.

Step 1: Apply concentrate to a frit. In the preferred method, said frit is a fritted glass. In this step, a concentrated substance or solution may be carefully applied onto a frit, with a preference for using a fritted glass. A fritted glass refers to a specialized type of glass that has been finely porous or sintered to create a network of small pores or channels within its structure. These pores may allow for enhanced surface area and improved flow characteristics. The concentrate, which could be a liquid or a viscous substance, may be applied onto the fritted glass surface using suitable techniques, such as pipetting, spraying, or controlled dispensing. The fritted glass may act as a support medium, providing a stable substrate for the application of the concentrate. The choice of using a fritted glass in this step offers several advantages. The porous structure of the fritted glass aids in distributing the concentrate evenly across its surface, allowing for efficient contact and interaction between the concentrate and the surrounding environment. The increased surface area provided by the porous structure also promotes enhanced absorption or filtration capabilities, depending on the specific application.

Step 2: Allow concentrate to absorb into interstices of said frit, preferably through application of heat to said concentrate and frit. In the preferred method, heat is applied via radiant heating from a proximal ohmic resistive heating element. In alternative methods, other heat sources may be used including flame or high-temperature air or other gas. In this step, after the concentrate has been applied to the frit in Step 1, the focus is on facilitating the absorption of the concentrate into the interstices or small spaces within the fritted glass structure. The preferred method involves the application of heat to aid in this absorption process, and specifically, the use of radiant heating from a nearby ohmic resistive heating element. Step 2 involves allowing the concentrate to absorb into the interstices of the fritted glass by applying heat. The preferred method may utilize radiant heating from a proximal ohmic resistive heating element, while alternative methods may involve different heat sources such as flame or high-temperature air or gas. The heat facilitates the absorption process, aiding in the distribution and integration of the concentrate within the frit structure.

Step 3: Continue heat application to induce vaporization of said concentrate to produce a vapor. Following the absorption of the concentrate into the interstices of the fritted glass, the process moves to the next stage of inducing vaporization. The application of heat is continued, maintaining the required temperature for the vaporization process. As the heat is applied, the absorbed concentrate within the fritted glass starts to reach its boiling or evaporation point. The increased temperature energizes the molecules of the concentrate, causing them to transition from the liquid or solid state to the gaseous state. This phase change results in the production of a vapor. The sustained heat application ensures that the absorbed concentrate is heated adequately to generate enough vapor. The vapor can consist of volatile compounds, active ingredients, or other substances present in the concentrate. The specific composition and characteristics of the vapor depend on the nature of the concentrate itself. It is important to note that the precise temperature and duration of heat application may vary based on factors such as the properties of the concentrate, desired vaporization rate, and the specific application requirements. The heat source used in Step 2, such as radiant heating from an ohmic resistive heating element or alternative methods mentioned previously, may continue to be employed to maintain the necessary heat levels.

Step 4: Evacuate said vapor. Once the vaporization of the concentrate has taken place and the vapor is generated, the next action is to evacuate or remove the vapor from the system. This step involves directing the vapor out of the immediate environment or extracting it for further processing or utilization. The method of evacuation can vary depending on the specific application or system setup. It may involve the use of fans, blowers, or vacuum systems to create a controlled airflow that carries the vapor away. The vapor may be directed through ducts or pipes to a designated area, such as a collection chamber or exhaust system. The purpose of evacuating the vapor is to ensure its proper containment or disposal, as well as to create a controlled environment for subsequent steps in the process. The removal of the vapor allows for further processing, analysis, or utilization of the vaporized components. It is important to note that the specifics of vapor evacuation can depend on factors such as the volume and characteristics of the vapor, the desired flow rate, and the safety considerations associated with the vaporized substances. Proper ventilation and safety measures should be implemented to handle any potentially hazardous vapors in accordance with relevant regulations and guidelines.

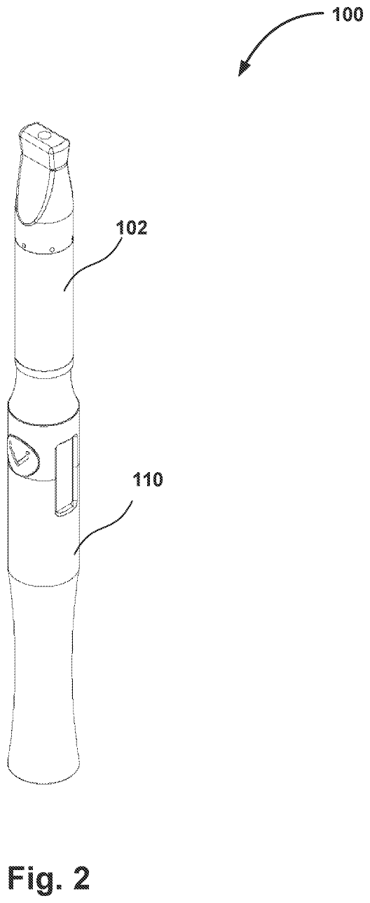

FIG. 2 is an isometric view of the vaporizer, which is adapted for performing the invented method. The vaporizer 100 consists of two main components: an atomizer 102 and a battery 110. The atomizer 102 engages to the battery 110 adapted to supply electrical current to the atomizer 102.

In an embodiment, the atomizer 102 is responsible for vaporizing the substance, while the battery 110 supplies the necessary electrical current to power the atomizer 102. The atomizer 102 is a crucial part of the vaporizer 100 that facilitates the vaporization process. It typically includes a heating element, such as a coil or a ceramic element, that is designed to reach the appropriate temperature for vaporizing the substance. When the battery 110 is activated, it supplies electrical current to the atomizer 102, causing the heating element to heat up. The substance to be vaporized, which can be a liquid or solid concentrate, is then introduced into the atomizer 102. The heat generated by the atomizer's heating element vaporizes the substance, converting it into a vapor or gas form that can be inhaled by the user. The battery 110 is responsible for powering the atomizer 102. It supplies the electrical current necessary for the heating element to reach the required temperature and initiate the vaporization process. The battery 110 is typically rechargeable and may be removable or integrated into the vaporizer 100 itself. The battery 110 may have various specifications, including voltage, capacity, and output capabilities, depending on the specific design, and intended use of the vaporizer 100. It can be charged using a compatible charging device, such as a USB cable or a dedicated charging dock. The interaction between the atomizer 102 and the battery 110 is critical for the proper functioning of the vaporizer 100. The battery 110 supplies the electrical power needed to activate the atomizer 102 and initiate the vaporization process. Without the battery 110, the atomizer 102 would not receive the necessary electrical current, rendering the vaporizer 100 inoperable.

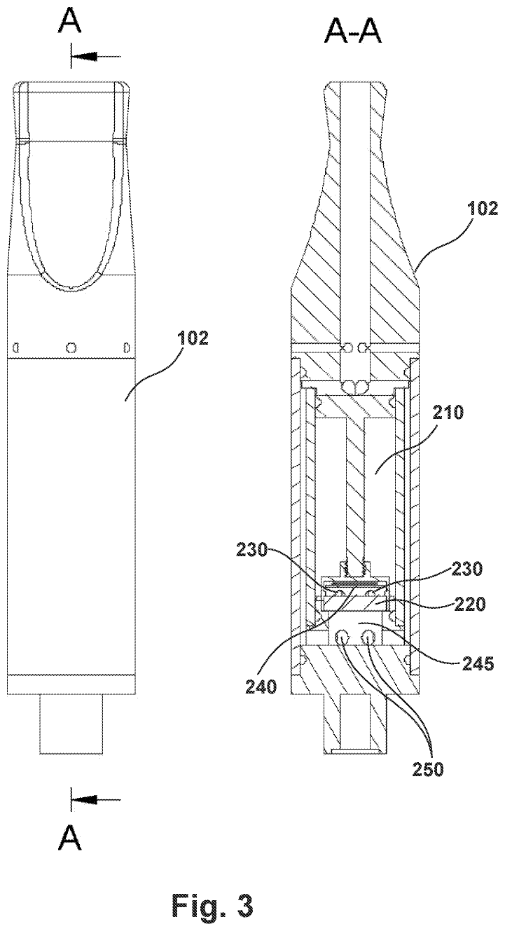

FIG. 3 is a front view and section view of the atomizer 102. The atomizer 102 is comprised of a concentrate reservoir volume 210 capable of containing some volume of concentrate, a concentrate vaporization assembly consisting of a frit filter 220 and a heat source or heating element 240 configured proximal to the frit 220, a supply port 230 through which concentrate may flow from the reservoir volume 210 to the frit 220, and a vapor collection and discharge assembly consisting of a vapor accumulation chamber 245 and a vapor evacuation channel 250.

In an embodiment, the concentrate reservoir volume 210 is a designated space within the atomizer 102 that can contain a certain volume of concentrate. It is designed to securely hold the concentrate, ensuring it remains contained and ready for vaporization. The reservoir volume 210 may have specific dimensions or capacity tailored to accommodate the desired amount of concentrate.

In an embodiment, the concentrate vaporization assembly within the atomizer 102 includes a frit filter 220 and a heat source or heating element 240. The frit filter 220 is positioned within the atomizer 102 to act as a filter or porous material through which the concentrate passes. The frit filter 220 helps distribute the concentrate evenly and aids in vaporization by increasing the surface area exposed to the heat source. The heat source or heating element 240 is configured proximal to the frit filter 220, typically positioned in close proximity to maximize heat transfer efficiency. It generates heat to raise the temperature of the concentrate and facilitate its vaporization. The heating element 240 may consist of a coil, a ceramic element, or another suitable heating mechanism capable of reaching the necessary temperature for vaporization.

In an embodiment, the supply port 230 serves as a conduit through which the concentrate flows from the reservoir volume 210 to the frit filter 220. It enables controlled delivery of the concentrate to the area where vaporization occurs. The design of the supply port 230 may include valves or other mechanisms to regulate the flow of concentrate and prevent leakage or spills.

In an embodiment, the vapor collection and discharge assembly may comprise a vapor accumulation chamber 245 and a vapor evacuation channel 250. After vaporization, the vapor produced from the concentrate accumulates in the vapor accumulation chamber 245. This chamber acts as a holding area, allowing the vapor to gather before it is directed for further use or disposal. The vapor evacuation channel 250 provides a pathway for the vapor to exit the atomizer. It connects the vapor accumulation chamber 245 to an external location, such as the mouthpiece or an outlet, where the vapor can be collected or directed for inhalation or other purposes. The design and placement of the vapor evacuation channel 250 ensure efficient and controlled discharge of the vapor from the atomizer.

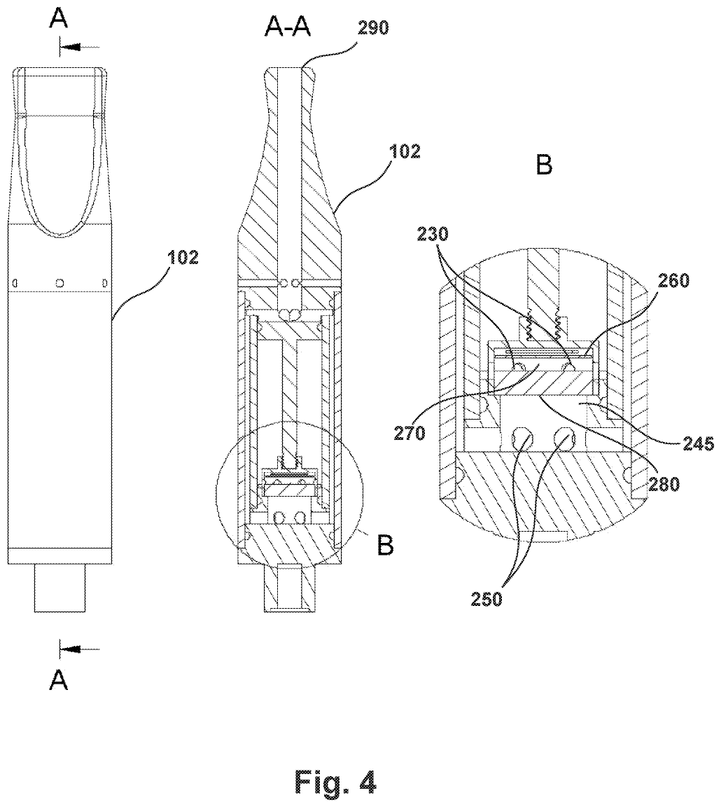

FIG. 4 shows front and section views of the atomizer and a detail view of the frit 220 and proximal components. In the preferred embodiment, the supply ports 230 are positioned such that concentrate freely flows to a concentrate preheating chamber 270. In the preferred embodiment, the preheating chamber 270 is a cylindrical reservoir volume formed by the frit 220 on its lower face, and the heating element 240 at its upper face. When the heat source 240 is operated, concentrate contained within the preheating chamber 270 will increase in temperature with a resulting reduction in viscosity. The reduced viscosity allows the concentrate to be readily absorbed into the interstices of the frit 220.

In the preferred embodiment, the design of the atomizer 102 may include the positioning of supply ports 230 in such a way that allows the concentrate to flow freely into a concentrate preheating chamber 270. This preheating chamber 270 is designed as a cylindrical reservoir volume, which is formed by the frit filter 220 on its lower face and the heating element 240 on its upper face. When the heat source or heating element 240 is activated, it generates heat within the preheating chamber 270. As a result, the concentrate contained within the preheating chamber 270 undergoes an increase in temperature. This rise in temperature leads to a reduction in the viscosity of the concentrate. The reduction in viscosity is a crucial aspect as it enables the concentrate to be readily absorbed into the interstices or small spaces within the frit filter 220. The frit filter, with its porous structure, provides a network of interconnecting channels or pores that can accommodate the concentrate in its vaporizable state. The reduced viscosity allows the concentrate to flow more easily and be absorbed into the frit filter 220, ensuring efficient distribution and integration within its porous structure. The preheating chamber 270, formed by the combination of the frit filter 220 and the heating element 240, acts as a controlled environment where the concentrate is subjected to heat. This controlled heating process increases the temperature of the concentrate and reduces its viscosity, making it more suitable for absorption into the interstices of the frit filter 220.

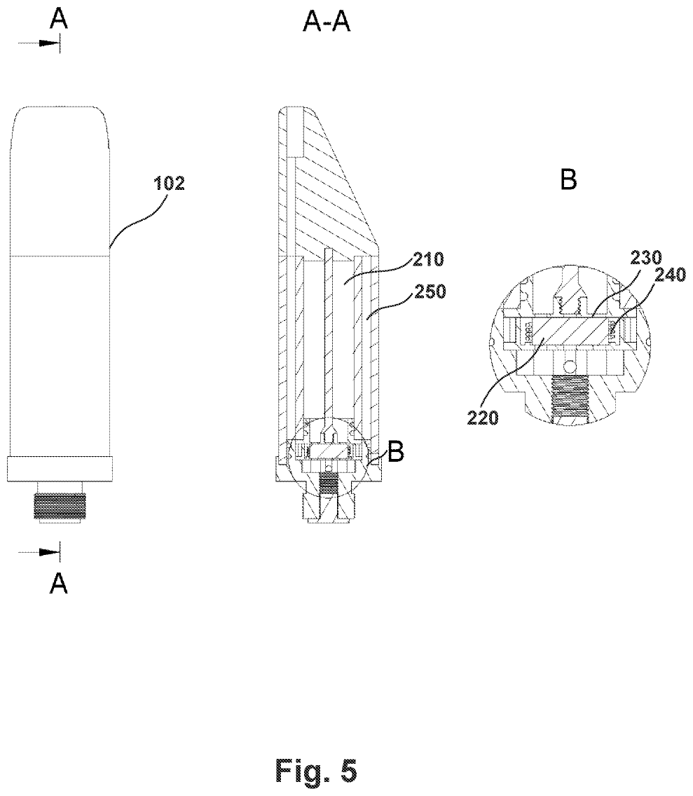

FIG. 5 shows an alternative embodiment of the atomizer 102. In this embodiment, the heating element 240 is a helical resistive element that is positioned proximally and coaxially with the frit 220. In the embodiment, the supply port 232 is in direct fluid communication with the frit 220. As concentrate is vaporized, it vaporized concentrate accumulates at the vapor accumulation chamber 245 (volume below the frit's lower surface 280) and evacuated through the evacuation channel 250, concentrate will flow from the reservoir volume 210 into the frit 220 until the frit 220 is resaturated with concentrate.

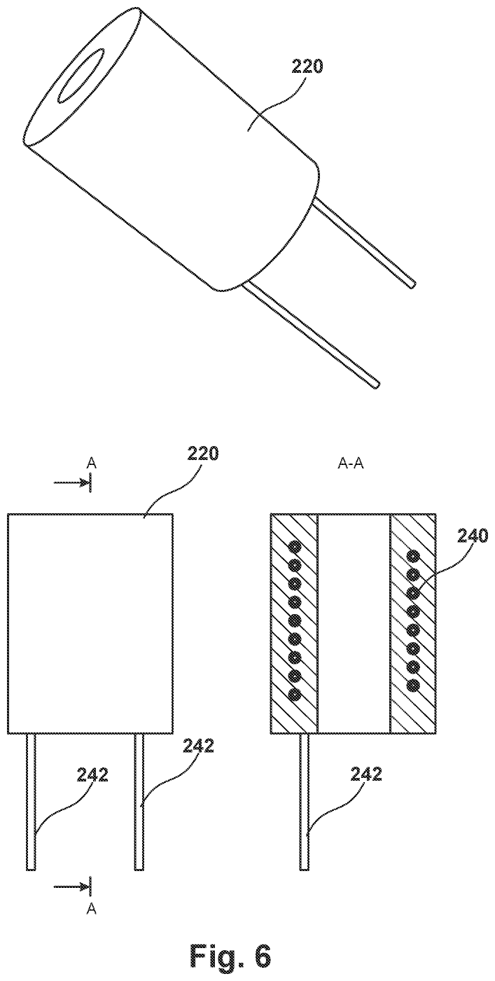

In the preferred embodiment, the heating source 240 is an ohmic heating element, which produces heat when electrical current is supplied by the battery 110. In the preferred embodiment, the heating element 240 is a coil, captured by a glass plate 260. In alternative embodiments, the heating element 240 may be potted, freely exposed within the preheating chamber 270, embedded within the preheating chamber 270 walls, embedded within the frit 220, or otherwise positioned proximal to or in contact with the frit 220 such that heat output from the heating element 240 is transferred to the frit 220. In an embodiment, the frit 220 is in the form of a hollow cylinder (as shown in FIG. 6) or another prismatic toroid. Fritted glass bodies are less susceptible to microscopic and macroscopic damage when a minimum feature radius is observed. In an embodiment, the fritted glass has a minimum feature radius of 1 mm. In alternative embodiments, the heat source 240 may be a flame or other heat source. The heating element 240 in the preferred embodiment is operable by a user through user control of the electrical current. In the preferred embodiment, the attached battery 110 will control current to the heat source 240, typically through a switch, in order to supply electrical current to the heating element 240. The heating element 240 will produce heat, which is transferred to the frit 220 and any absorbed concentrate. The heated concentrate will vaporize. Vaporized concentrate will freely flow through the frit 220 and exit through the frit's lower surface 280 and accumulate into the vapor accumulation chamber 245 and then get evacuated through the evacuation channel 250 which is in fluid communication with the vapor accumulation chamber 245. The resulting vapor may be evacuated from the device 100 through the evacuation channel 250 by means of application of a pressure differential, said pressure differential typically produced by a user inhaling through the evacuation channel 250 at the egress port 290.

As concentrate contained within the frit 220 is vaporized, gravity and capillary action will cause concentrate to flow from the concentrate reservoir 210 through the supply port 230 to resaturate the frit 220. Resaturation is aided by preheating concentrate contained in the preheating chamber 270. Resaturation of the frit 220 is further aided by the arrangement of the heating element 240, the preheating chamber 270, and the frit 220.

FIG. 6 shows a perspective, front, and section views of an alternative embodiment of the frit having an embedded heating element. In the embodiment, the frit 220 is generally cylindrical in shape and is hollow inside. The heating element 240 has leads 242 that, when connected to the cathode and anode of a battery (such as the battery 110) form an electrical circuit. The heating element 240 is embedded within the volume of the frit 220.

### Preferred Method of Use

In the preferred method of use, the concentrate is of sufficiently low viscosity that it will readily flow toward gravity. The heating element 240 is activated by user control of a switch on the battery 110, which will cause concentrate contained within the frit 220 to vaporize, and the user will inhale resulting vapor by inhaling at the egress port 290 of the evacuation channel 250.

In summary, the vaporizer 100 includes the atomizer 102, the battery 110 supplying electrical current to the atomizer 102, and a user-activated switch to activate the heating element for vaporization. The vaporizer 100 also includes a vapor accumulation chamber, a vapor evacuation channel, and an egress port for the user to inhale the resulting vapor. Additionally, the battery may be rechargeable with a charging port.

The described atomizer (102) comprises a concentrate reservoir volume (210) for holding the concentrate, a concentrate vaporization assembly with a frit filter (220) that absorbs the concentrate and a heating element (240) located near the frit filter to heat both the filter and the absorbed concentrate. It also includes a vapor collection and discharge assembly with a vapor accumulation chamber (245) connected to the frit filter and a vapor evacuation channel (250) that leads to an egress port (290). The atomizer has a supply port (230) facilitating the free flow of the concentrate into a concentrate preheating chamber (270) formed by the frit filter and the heating element. Further the device includes specific features such as the use of an ohmic resistive heating element, a porous ceramic material for the frit filter, a control module for temperature regulation, and options for the frit filter being made of sintered glass or sintered quartz glass. The heating element may be embedded within the frit filter, and the frit filter may have a hollow cylindrical shape with a minimum feature radius of 1 mm. The concentrate preheating chamber is positioned proximal to the frit filter, and the heating element can be helical in shape and coaxially positioned with the frit filter or captured by a glass plate or potted and exposed or embedded within the concentrate preheating chamber.

While preferred and alternate embodiments have been illustrated and described, as noted above, many changes can be made without departing from the spirit and scope of the present disclosure. Accordingly, the scope is not limited by the disclosure of these preferred and alternate embodiments. Instead, the scope of the present disclosure is to be determined entirely by reference to the claims. Insofar as the description above and the accompanying drawings (if any) disclose any additional subject matter that is not within the scope of the claims below, the inventions are not dedicated to the public and Applicant hereby reserves the right to file one or more applications to claim such additional inventions.

The reader's attention is directed to all papers and documents which are filed concurrently with this specification, and which are open to public inspection with this specification, and the contents of all such papers and documents are incorporated herein by reference.

All the features disclosed in this specification (including any accompanying claims, abstract, and drawings) may be replaced by alternative features serving the same, equivalent or similar purpose, unless expressly stated otherwise. Thus, unless expressly stated otherwise, each feature disclosed is one example of a generic series of equivalent or similar features.

Any element in a claim that does not explicitly state “means for” performing a specified function, or “step for” performing a specific function is not to be interpreted as a “means” or “step” clause as specified in 35. U.S.C. § 112 ¶6. In particular, the use of “step of” in the claims herein is not intended to invoke the provisions of U.S.C. § 112 ¶16.

What is claimed is:

1. An atomizer (102) comprising:
a concentrate reservoir volume (210) configured for holding concentrate;
a concentrate vaporization assembly comprising:
a frit filter (220) designed to absorb the concentrate from the concentrate reservoir volume (210); and
a heating element (240) located proximal to the frit filter (220) to heat the frit filter (220) and the absorbed concentrate;
a vapor collection and discharge assembly comprising:
a vapor accumulation chamber (245) in fluid communication with the frit filter (220); and
a vapor evacuation channel (250) connecting the vapor accumulation chamber (245) to an egress port (290); and
a supply port (230) providing direct fluid communication between the frit filter (220) and the concentrate reservoir volume (210), facilitating the free flow of the concentrate into a concentrate preheating chamber (270) formed by the frit filter (220) on its lower face and the heating element (240) on its upper face.

2. The atomizer (102) of claim 1, wherein the heating element (240) is an ohmic resistive heating element.

3. The atomizer (102) of claim 1, wherein the frit filter (220) is made of a porous ceramic material.

4. The atomizer (102) of claim 1, further comprising a control module configured to regulate the temperature of the heating element (240) based on user preferences.

5. The atomizer (102) of claim 1, wherein the frit filter (220) is sintered glass.

6. The atomizer (102) of claim 5, wherein the frit filter (220) is sintered quartz glass.

7. The atomizer (102) of claim 1, wherein the heating element (240) is embedded within the frit filter (220).

8. The atomizer (102) of claim 7, wherein the frit filter (220) is in a shape of a hollow cylinder.

9. The atomizer (102) of claim 8, wherein the frit filter (220) has a minimum feature radius of 1 mm.

10. The atomizer (100) of claim 1, wherein the concentrate preheating chamber (270) is in proximal to the frit filter (220) for receiving the concentrate from the concentrate reservoir volume (210).

11. The atomizer (102) of claim 1, wherein the heating element (240) is helical in shape and positioned proximally and coaxially with the frit filter (220).

12. The atomizer (102) of claim 1, wherein the heating element (240) is configured as a coil, captured by a glass plate (260).

13. The atomizer (102) of claim 1, wherein the heating element (240) is potted and freely exposed or embedded within the concentrate preheating chamber (270) such that heat output from the heating element (240) is transferred to the frit filter (220).

14. A vaporizer (100) comprising:
an atomizer (102);
a battery (110) adapted to supply electrical current to the atomizer (100);
wherein, the atomizer (100) comprising:
a concentrate reservoir volume (210) configured for holding concentrate, wherein the concentrate reservoir volume (210) is in fluid communication with a concentrate vaporization assembly, the concentrate vaporization assembly comprising:
a frit filter (220) adapted to absorb concentrate from the concentrate reservoir volume (210), and
a heating element (240) proximal to the frit filter (220), and adapted to heat the frit filter (220) and absorbed concentrate; and
a vapor collection and discharge assembly comprising:
a vapor accumulation chamber (245) in fluid communication with the frit filter, and
a vapor evacuation channel (250) in fluid communication with the vapor accumulation chamber and in fluid communication with an egress port (290), wherein the heating element (240) is activated by a user control of a switch on the battery (110), which causes the concentrate contained within the frit filter (220) to vaporize, and the user inhales resulting vapor by inhaling at the egress port (290) of the vapor evacuation channel (250), and
a supply port (230),
wherein the supply port (230) is in direct fluid communication with the frit filter (220) positioned to allow free flow of the concentrate from the concentrate reservoir volume (210) into a concentrate preheating chamber (270), and wherein the concentrate preheating chamber (270) is a cylindrical reservoir volume formed by the frit filter (220) on its lower face and the heating element (240) at its upper face.

15. The vaporizer (100) of claim 14, wherein the frit filter (220) is made of sintered glass or sintered quartz glass.

16. The vaporizer (100)) of claim 14, wherein the heating element (240) is embedded within the frit filter (220).

17. The vaporizer (100) of claim 14, wherein the frit filter (220) is a hollow cylinder shape with a minimum feature radius of 1 mm.

18. The vaporizer (100) of claim 14, wherein the concentrate preheating chamber (270) is located proximal to the frit filter (220) to receive the concentrate from the concentrate reservoir volume (210).

19. The vaporizer (100) of claim 14, wherein the heating element (240) has a helical shape and is positioned proximally and coaxially with the frit filter (220), or is a coil captured by a glass plate (260), or is potted and freely exposed or embedded within the concentrate preheating chamber (270) to transfer heat to the frit filter (220).

20. The vaporizer (100) of claim 14, wherein the battery (110) is rechargeable and includes a charging port for recharging the battery's power supply.
